(19) United States
(12) Patent Application Publication
(10) Pub. No.: US 2022/0380756 A1
(43) Pub. Date: Dec. 1, 2022

(54) METHODS AND COMPOSITIONS FOR TREATING THALASSEMIA OR SICKLE CELL DISEASE

(71) Applicant: ASC THERAPEUTICS INC., Milpitas, CA (US)

(72) Inventors: Xuefeng WANG, Fremont, CA (US); Ling-jie KONG, Union City, CA (US); H. Steve ZHANG, Moraga, CA (US); Ruby Yanru TSAI, San Jose, CA (US)

(21) Appl. No.: 17/623,255

(22) PCT Filed: Jun. 29, 2020
(86) PCT No.: PCT/US2020/040199

§ 371 Date: Dec. 28, 2021

Related U.S. Application Data
(60) Provisional application No. 62/867,877, filed on Jun. 28, 2019.

(51) Int. Cl. C12N 15/11; C12N 9/22; C12N 15/90; A61K 35/28; C07K 14/805

(52) U.S. Cl. CPC C12N 15/11; C12N 9/22; C12N 15/907; A61K 35/28; C07K 14/805; C12N 2310/20; A61K 38/00

(57) ABSTRACT

Provided herein are methods and compositions for treating genetic blood cell diseases, e.g. sickle cell disease and thalassemia, by correcting genetic mutation or inserting exogenous globin gene using CRISPR/Cas system.

### CROSS-REFERENCE TO RELATED APPLICATIONS

This application claims priority to U.S. Provisional Application No. 62/867,877, filed Jun. 28, 2019, the disclosure of which is incorporated herein by reference.

### SEQUENCE LISTING

The present application contains a Sequence Listing which has been submitted electronically in ASCII format and is hereby incorporated by reference in its entirety. Said ASCII copy, created on Jun. 28, 2020, is named “044903-8026WO01-Sequence Listing_ST25” and is 5k bytes in size.

### FIELD OF THE INVENTION

The present invention generally relates to genome engineering. More specifically, the present invention relates to methods and compositions for treating genetic diseases of blood cells.

### BACKGROUND

Hemoglobin (Hb) is the iron-containing oxygen-transport metalloprotein in the red blood cells (RBC) of almost all vertebrates. In mammals, hemoglobin makes up about 96% of the RBC's dry content by weight, and around 35% of the total content including water. In humans, the hemoglobin molecule is an assembly of four globular protein subunits, each composed of a globin protein tightly associated with a prosthetic heme group. Genetic diseases of blood cells that result in hemoglobin abnormalities, including sickle cell disease (SCD) and thalassemia, affect hundreds of thousands worldwide.

SCD is caused by a single-nucleotide polymorphism (SNP) in the seventh codon of the gene for beta-globin (HBB), one of two globins that make up the major adult form of hemoglobin. The resulting glutamate-to-valine substitution renders hemoglobin prone to polymerization under hypoxic conditions, producing characteristic “sickle”-shaped red blood cells, which has a markedly reduced life span in the bloodstream, damages the vasculature, and causes vaso-occulusion.

Thalassemia is an inherited blood disorder characterized by abnormal hemoglobin production. Symptoms of thalassemia depend on the type and include mild to severe anemia, bone problems, enlarged spleen, yellowish skin and dark urine. Beta thalassemia is due to mutations in the HBB gene on chromosome 11, inherited in an autosomal, recessive fashion.

While the genetic and molecular basis of SCD and thalassemia have been understood for decades, curative treatments have been lagged. Therefore, there is a continuing need to develop new methods and compositions to treat SCD and thalassemia.

### SUMMARY OF THE INVENTION

In one aspect, the present disclosure provides a guide RNA or a nucleic acid encoding the same, which can be used to treat sickle cell disease (SCD) or thalassemia. In one aspect, the guide RNA described herein targets a site as shown in any of SEQ ID NOS: 1, 8, 15, 22, 29, 36 and 47 in a beta-globin gene. In certain embodiments, the guide RNA comprises a polynucleotide sequence having at least 95%, at least 96%, at least 97%, at least 98 percent, at least 99% identity of any of SEQ ID NOS: 2-7, 9-14, 16-21, 23-28, 30-35, 37-42, and 48-53. In certain embodiments, the guide RNA comprises a polynucleotide sequence having 1, 2, 3, 4 or 5 nucleotide difference from any of SEQ ID NOS: 2-7, 9-14, 16-21, 23-28, 30-35, 37-42, and 48-53. In certain embodiments, the guide RNA comprises a polynucleotide sequence of any of SEQ ID NOS: 2-7, 9-14, 16-21, 23-28, 30-35, 37-42, and 48-53.

In certain embodiments, the guide RNA described herein is a single guide RNA (sgRNA). In certain embodiments, the guide RNA described herein is made up of CRISPR RNA (crRNA) and trans-activating RNA (tracrRNA). In certain embodiments, the guide RNA is for Cas9 nuclease. In certain embodiments, the guide RNA is for Cpf1 nuclease.

In another aspect, the present disclosure provides a composition comprising a CRISPR/Cas nuclease or a nucleic acid encoding the same; and the guide RNA described herein or a nucleic acid encoding the same, wherein the CRISPR/Cas nuclease is associated with the guide RNA and is capable of cleaving the beta-globin gene. In certain embodiments, the CRISPR/Cas nuclease is a Cas9 nuclease. In certain embodiments, the CRISPR/Cas nuclease is a Cpf1 nuclease.

In another aspect, the present disclosure provides an isolated mammalian cell comprising the composition described herein. In certain embodiments, the isolated mammalian cell is a stem cell. In certain embodiments, the stem cell is a hematopoietic stem/progenitor cell (HSPC). In certain embodiments, the isolated mammalian cell is obtained from a subject having sickle cell disease or thalassemia. In certain embodiments, the isolated mammalian cell described herein further comprises a transgene encoding a wildtype beta-globin polypeptide.

In another aspect, the present disclosure provides a method of modifying an isolated mammalian cell. In certain embodiments, the method comprises introducing to the mammalian cell the composition described herein, wherein the CRISPR/Cas nuclease cleaves the beta-globin gene in the mammalian cell. In certain embodiments, the mammalian cell is obtained from a subject having sickle cell disease or thalassemia, and the method further comprises introducing to the mammalian cell a nucleic acid comprising a transgene encoding a wildtype beta-globin polypeptide such that the transgene is inserted to the target site. In certain embodiments, the nucleic acid is a single-strand DNA or double-strand DNA. In certain embodiments, the nucleic acid is contained in a virus vector. In certain embodiments, the virus is adeno-associated virus (AAV).

In yet another aspect, the present disclosure provides a method of treating a sickle cell disease or thalassemia in a subject. In certain embodiments, the method comprises administering to the subject the mammalian cell described herein. In certain embodiments, the mammalian cell is obtained from the subject. In certain embodiments, the method comprises administering to the subject the composition described herein, wherein the CRISPR/Cas nuclease cleaves the beta-globin gene in a cell of the subject.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates the exemplary gRNAs targeting HBB gene as described herein.

FIG. 2 illustrates the exemplary sequences of crRNA, tracrRNA, sgRNA for SpCas9 and gRNA for Cpf1.

FIG. 3 illustrates the guide sequences of exemplary gRNAs as described herein.

FIG. 4 illustrates the cleavage efficiency of SpCas9 gRNAs targeting HBB exon 1 as measured by NGS.

FIG. 5 illustrates the cleavage efficiency of Cpf1 (Cas12a) gRNAs targeting HBB exon 1 and intron 1 as measured by NGS.

FIG. 6 illustrates the correction of the sickle mutation in the SCD cell lines using gRNAs described herein.

FIG. 7 illustrates a schematic of knock-in of WT HBB gene into intron 1 of HBB in the β°/β° thalassemia cell line and the detection thereof using ddPCR.

FIG. 8 illustrates the detection of knock-in of WT HBB gene into intron 1 of HBB in the β°/β° thalassemia cell line using gRNAs described herein.

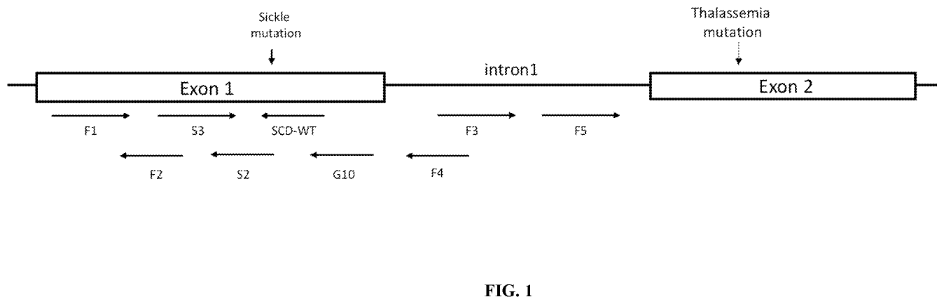

### DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

As used herein, the singular forms “a”, “an” and “the” include plural references unless the context clearly dictates otherwise.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

A “cell”, as used herein, can be any eukaryotic cell(s), for example a mammalian cell or cell line, including COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces. Primary cells can also be edited as described herein, including but not limited to fibroblasts, blood cells (e.g., red blood cells, white blood cells), liver cells, kidney cells, neural cells, and the like. Suitable cells also include stem cells such as, by way of example, embryonic stem cells, induced pluripotent stem cells (iPSCs), hematopoietic stem cells, neuronal stem cells and mesenchymal stem cells. In other aspects, genetically modified blood cell precursors (hematopoietic stem/progenitor cells known as “HSPCs”) are given in a bone marrow transplant and the HSPCs differentiate and mature in vivo.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends.

As used herein, the terms “chimeric RNA” or “single guide RNA” refers to the polynucleotide sequence comprising the guide sequence, the tracr sequence and the tracr mate sequence. The term “guide sequence” refers to the about 10-30 (10, 11, 12, 13, 14, 15, 16, 17, 18, 19, 20, 21, 22, 23, 24, 25, 26, 27, 28, 29, 30) base pair sequence within the guide RNA that specifies the target site.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “subject” or “individual” or “animal” or “patient” as used herein refers to human or non-human animal, including a mammal or a primate, in need of diagnosis, prognosis, amelioration, prevention and/or treatment of a disease or disorder such as viral infection or tumor. Mammalian subjects include humans, domestic animals, farm animals, and zoo, sports, or pet animals such as dogs, cats, guinea pigs, rabbits, rats, mice, horses, swine, cows, bears, and so on.

In the context of formation of a CRISPR complex, “target” refers to a guide sequence (that is, gRNA) designed to have complementarity to a genomic region (that is, a target sequence), where hybridization between the genomic region and a guide RNA promotes the formation of a CRISPR complex. The terms “complementarity” or “complementary” are used in reference to polynucleotides (i.e., a sequence of nucleotides) related by the base-pairing rules. Complementarity may be “partial,” in which only some of the nucleic acids' bases are matched according to the base pairing rules (e.g., 5, 6, 7, 8, 9, 10 out of 10 being 50%, 60%, 70%, 80%, 90%, and 100% complementary), or there may be “complete” or “total” complementarity between the nucleic acids. The degree of complementarity between nucleic acid strands has significant effects on the efficiency and strength of their hybridization to one another.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

### Genetic Diseases of Blood Cells

The present disclosure in one aspect relates to methods and compositions for treating genetic diseases of blood cells. In certain embodiments, the genetic diseases of blood cells involve the defects of hemoglobin gene.

Hemoglobin is a heterotetramer comprising two α-like globin chains and two β-like globin chains and 4 heme groups. In adult humans, the α2β2 tetramer is referred to as Hemoglobin A (HbA) or adult hemoglobin. Typically, the alpha and beta globin chains are synthesized in an approximate 1:1 ratio and this ratio seems to be critical in terms of hemoglobin and red blood cell stabilization. In fact, in some cases where one type of the globin genes is inadequately expressed, reducing expression (e.g. using a specific siRNA) of the other type of globin, restoring this 1:1 ratio, alleviates some aspects of the mutant cellular phenotype (see Voon et al., Haematologica (2008) 93(8):1288). In a developing fetus, a different form of hemoglobin, fetal hemoglobin (HbF) is produced which has a higher binding affinity for oxygen than Hemoglobin A such that oxygen can be delivered to the baby's system via the mother's blood stream. Fetal hemoglobin also contains two α globin chains, but instead of the adult β-globin chains, it has two fetal γ-globin chains (α2γ2). At approximately 30 weeks of gestation, the synthesis of γ-globin in the fetus starts to drop while the production of β-globin increases. By approximately 10 months of age, the newborn's hemoglobin is nearly all α2β2 although some HbF persists into adulthood (approximately 1-3% of total hemoglobin). Human beta-globin gene (HBB) has a USCS Genome Brower location of chr11:5246696-5248301 and a GenBank Accession Reference No: NM_000518.

Genetic defects in the sequences encoding the hemoglobin chains can be responsible for a number of diseases known as hemoglobinopathies, including sickle cell disease and thalassemia.

Sickle cell disease (SCD) is a recessive genetic disorder that affects at least 90,000 individuals in the United States and hundreds of thousands worldwide. There appears to be a benefit of sickle cell heterozygosity for protection against malaria, so this trait may have been selected for over time, such that it is estimated that in sub-Saharan Africa, one third of the population has the sickle cell trait. Sickle cell disease is caused by a mutation in the β-globin gene in which valine is substituted for glutamic acid at amino acid #6 (a GAG to GTG at the DNA level), where the resultant hemoglobin is referred to as “hemoglobin S” or “HbS.” Under lower oxygen conditions, the deoxy form of HbS exposes a hydrophobic patch on the protein between the E and F helices. The hydrophobic residues of the valine at position 6 of the beta chain in hemoglobin are able to associate with the hydrophobic patch, causing HbS molecules to aggregate and form fibrous precipitates. These aggregates in turn cause the abnormality or ‘sickling’ of the red blood cells (RBCs), resulting in a loss of flexibility of the cells. The sickling RBCs are no longer able to squeeze into the capillary beds and can result in vaso-occlusive crisis in sickle cell patients. In addition, sickled RBCs are more fragile than normal RBCs, and tend towards hemolysis, eventually leading to anemia in the patient.

Thalassemia is a disease relating to hemoglobin and typically involve a reduced expression of globin chains. This can occur through mutations in the regulatory regions of the genes or from a mutation in a globin coding sequence that results in reduced expression. There are two main types of thalassemia, alpha thalassemia and beta thalassemia, due to impaired production of alpha and beta globin chain, respectively. Alpha thalassemia is associated with people of Western Africa and South Asian descent, and may confer malarial resistance. Beta thalassemia is associated with people of Mediterranean descent, typically from Greece and the coastal areas of Turkey and Italy. Treatment of thalassemia usually involves blood transfusions and iron chelation therapy. Bone marrow transplants are also being used for treatment of people with severe thalassemia if an appropriate donor can be identified, but this procedure can have significant risks.

In one aspect, the present disclosure provides methods and compositions for treating genetic diseases for blood cells.

### The CRISPR/Cas System

In certain embodiments, the methods and compositions provided herein involve genome engineering, in particular in the hemoglobin gene, using CRISPR (Clustered Regularly Interspaced Short Palindromic Repeats)/Cas system.

CRISPR/Cas system was originally found as an RNA-mediated genome defense pathway in the prokaryotic cells (see Godde and Bickerton, J. Mol. Evol. (2006) 62: 718-729; Lillestol et al., Archaea (2006) 2: 59-72; Makarova et al., Biol. Direct (2006) 1: 7; Sorek et al., Nat. Rev. Microbiol. (2008) 6: 181-186). The pathway is proposed arise from two evolutionarily and often physically linked gene loci: the CRISPR locus, which encodes RNA components of the system, and the Cas (CRISPR associated) locus, which encodes protein (Jansen et al., Mol. Microbiol. (2002) 43: 1565-1575; Makarova et al., Nucleic Acids Res. (2002) 30: 482-496; Makarova et al., Biol. Direct (2006) 1: 7; Haft et al., PLoS Comput. Biol. (2005) 1: e60). CRISPR loci in microbial hosts contain a combination of CRISPR-associated (Cas) genes as well as non-coding RNA elements capable of programming the specificity of the CRISPR-mediated nucleic acid cleavage. The Cas genes are often associated with CRISPR repeat-spacer arrays. More than forty different Cas protein families have been described.

CRISPR/Cas systems fall into two classes: Class 1 systems use a complex of multiple Cas proteins to degrade foreign nucleic acids, while Class 2 systems use a single large Cas protein for the same purpose. Class 1 is divided into types I, III and IV; class 2 is divided into types II, V and VI. The six types have been divided into 19 subtypes.

The Type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer.

Type II CRISPR systems have been found in many different bacteria. BLAST searches on publicly available genomes by Fonfara et al., (Nuc Acid Res (2013) 42(4):2377-2590) found Cas9 orthologs in 347 species of bacteria. Additionally, this group demonstrated in vitro CRISPR/Cas cleavage of a DNA target using Cas9 orthologs from S. pyogenes, S. mutans, S. therophilus, C. jejuni, N. meningitides, P. multocida and F. novicida. Thus, the term “Cas9” as used herein refers to an RNA guided DNA nuclease comprising a DNA binding domain and two nuclease domains, where the gene encoding the Cas9 may be derived from any suitable bacteria.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The Cas9 endonuclease can be engineered such that only one of the nuclease domains is functional, creating a Cas nickase (see Jinek et al. Science (2012) 337:816). Nickases can be generated by specific mutation of amino acids in the catalytic domain of the enzyme, or by truncation of part or all of the domain such that it is no longer functional. Since Cas9 comprises two nuclease domains, this approach may be taken on either domain. A double strand break can be achieved in the target DNA by the use of two such Cas9 nickases. The nickases will each cleave one strand of the DNA and the use of two will create a double strand break.

The requirement of the crRNA-tracrRNA complex can be avoided by use of an engineered “single-guide RNA” (sgRNA) that comprises the hairpin normally formed by the annealing of the crRNA and the tracrRNA (see Jinek et al. Science (2012) 337:816 and Cong et al. Sciencexpress (2013) 10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing in eukaryotic cells.

Cpf1, also known as Cas12a, is another Cas endonuclease that has been used for genome engineering. Initially characterized from the Prevotella and Francisella bacterium, Cpf1 shows several differences from Cas9 including: causing a “staggered” cut in double stranded DNA as opposed to the “blunt” cut produced by Cas9; relying on a “T rich” PAM while Cas9 relies on “G rich” PAM; and requiring only a CRISPR RNA (crRNA) for successful targeting while Cas9 requires both crRNA and tracrRNA.

### Guide RNA of CRISPR/Cas System

Along with Cas endonuclease, the CRISPR/Cas system used for genome engineering requires a guide RNA that guides the Cas endonuclease to a target nuclei acid.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al. Sciencexpress (2013) 1/10.1126/science 1231143). In certain embodiments, the tracrRNA and pre-crRNAs are provided via separate expression constructs or as separate RNAs. An exemplary crRNA for SpCas9 is illustrated in SEQ ID NO: 54 wherein the nucleotides labeled as “N” represent the guide sequence. An exemplary tracrRNA for SpCas9 is illustrated in SEQ ID NO: 55. In certain embodiments, a single guide RNA is constructed where an engineered mature crRNA (conferring target specificity) is fused to a tracrRNA (supplying interaction with the Cas9) to create a chimeric crRNA-tracrRNA hybrid. (see Jinek et al. Science (2012) 337:816 and see Cong et al. Sciencexpress (2013) 1/10.1126/science 1231143). An exemplary sgRNA with scaffold for SpCas9 is illustrated in SEQ ID NO: 56.

A guide RNA can be designed using any known software in the art, such as Target Finder, E-CRISPR, CasFinder, and CRISPR Optimal Target Finder. Typically, the guide RNA used for CRISPR/Cas9 system contains an approximately 15 to 30 base sequence complementary to a target nucleic acid (e.g., DNA) that is followed by a protospacer-adjacent motif (PAM) in the form NGG. Alternative PAM sequences may also be utilized, where a PAM sequence can be NAG as an alternative to NGG (Hsu et al. Nature Biotech (2013) doi:10.1038/nbt.2647) using a S. pyogenes Cas9. Additional PAM sequences may also include those lacking the initial G (Sander and Joung Nature Biotech (2014) 32(4):347). In addition to the S. pyogenes encoded Cas9 PAM sequences, other PAM sequences can be used that are specific for Cas9 proteins from other bacterial sources. For example, the PAM sequences shown below are specific for these Cas9 proteins:

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |

In a sgRNA, the complementarity region is fused to a tracrRNA portion (see Hsu et al., (2013) Nature Biotech doi:10.1038/nbt.2647), which may be of 67 to 85 nucleotides. Truncated sgRNAs may also be used (see Fu et al. Nature Biotech (2014) 32(3): 279).

Guide RNAs that bind to Cpf1 has been described, e.g., in U.S. Pat. No. 10,669,540B2 to Zhang et al., the disclosure of which is incorporated herein via reference. Unlike the Cas9 system, Cpf1 uses a single guide RNA. The PAM sequence for Cpf1 is TTTV, where V is A, C or G (Zetsche B, et al. (2015) Cell, 163:759-771), and is located 5′ to the gRNA target sequence. An exemplary gRNA with scaffold for Cpf1 is illustrated in SEQ ID NO: 57.

### Transgene

After a Cas endonuclease, with the help of a guide RNA, introduces a cleavage at a target nucleic acid, an exogenous sequence (also called a “transgene”) can be inserted at the cleavage site. The transgene can be inserted via homologous recombination. In such cases, a donor nucleic acid can contain the transgene sequence flanked by two regions of homology to allow for efficient homologous recombination at the location of interest. Alternatively, the transgene can be inserted via non-homologous end joining (NHEJ). In such cases, the donor nucleic acid may have no regions of homology to the targeted location in the DNA. Additionally, the donor nucleic acid can comprise sequences of a vector molecule.

The donor nucleic acid can be DNA or RNA, single-stranded and/or double-stranded and can be introduced into a cell in linear or circular form. See, e.g., U.S. Patent Publication Nos. 2010/0047805 and 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. Proc. Natl. Acad. Sci. USA (1987) 84:4959-4963; Nehls et al. Science (1996) 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

The transgene can be introduced into a cell as part of a vector molecule having additional sequences such as, for example, replication origins, promoters and genes encoding antibiotic resistance. Moreover, the transgene can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

In certain embodiments, the transgene is inserted so that its expression is driven by the endogenous promoter at the integration site, namely the promoter that drives expression of the endogenous gene into which the transgene is inserted (e.g., globin, AAVS1, etc.). In certain embodiments, the donor nucleic acid can comprise a promoter and/or enhancer, for example a constitutive promoter or an inducible or tissue specific promoter, which drives the expression of the transgene after insertion to the target site.

The transgene may be inserted into an endogenous gene such that all, some or none of the endogenous gene is expressed. In certain embodiments, the transgene is integrated into any endogenous locus, for example a safe-harbor locus. In certain embodiments, the donor nucleic acid may also include transcriptional or translational regulatory sequences, for example, promoters, enhancers, insulators, internal ribosome entry sites, sequences encoding 2A peptides and/or polyadenylation signals.

### Introducing to the Cells

The Cas endonuclease or the nucleic acid encoding the Cas endonuclease, the guide RNA, the donor nucleic acid containing the transgene may be introduced to a cell in vivo or ex vivo by any suitable means.

Methods of introducing Cas endonucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Cas endonucleases, guide RNAs and/or donor nucleic acid as described herein may also be introduced using vectors containing sequences encoding one or more of the CRISPR/Cas system(s). Any vector systems may be used including, but not limited to, plasmid vectors, DNA minicircles, retroviral vectors, lentiviral vectors, adenovirus vectors, poxvirus vectors; herpesvirus vectors and adeno-associated virus vectors, etc., and combinations thereof. See, also, U.S. Pat. Nos. 6,534,261; 6,607,882; 6,824,978; 6,933,113; 6,979,539; 7,013,219; and 7,163,824, and U.S. Patent Publication No. 2014/0335063, incorporated by reference herein in their entireties. It can be understood that the Cas endonucleases, guide RNAs and the donor nucleic acid may be carried on the same vector or on different vectors.

Conventional viral and non-viral based gene transfer methods can be used to introduce nucleic acids encoding Cas endonucleases, guide RNAs and donor nucleic acid in cells (e.g., mammalian cells) and target tissues. Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science (1992) 256:808-813; Nabel & Felgner, TIBTECH (1993) 11:211-217; Mitani & Caskey, TIBTECH (1993) 11:162-166; Dillon, TIBTECH (1993) 11:167-175; Miller, Nature (1992) 357:455-460; Van Brunt, Biotechnology (1988) 6(10):1149-1154; Vigne, Restorative Neurology and Neuroscience (1995) 8:35-36; Kremer & Perricaudet, British Medical Bulletin (1995) 51(1):31-44; Haddada et al., in Current Topics in Microbiology and Immunology (1995) Doerfler and Bohm (eds.); and Yu et al., Gene Therapy (1994) 1:13-26.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

The use of RNA or DNA viral based systems for the delivery of nucleic acids encoding engineered CRISPR/Cas systems take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of CRISPR/Cas systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In certain embodiments, where transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology (1987) 160:38-47; U.S. Pat. No. 4,797,368; International Patent Publication No. WO 93/24641; Kotin, Human Gene Therapy (1994) 5:793-801; Muzyczka, J. Clin. Invest. (1994) 94:1351. Construction of recombinant AAV vectors are described in a number of publications, including U.S. Pat. No. 5,173,414; Tratschin et al., Mol. Cell. Biol. (1985) 5:3251-3260; Tratschin, et al., Mol. Cell. Biol. (1984) 4:2072-2081; Hermonat & Muzyczka, PNAS (1984) 81:6466-6470; and Samulski et al., J. Virol. (1989) 63:03822-3828.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences.

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing the Cas endonucleases, guide RNAs and/or donor nucleic acid can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It can be understood that the Cas endonuclease-encoding sequences, the guide RNAs and the donor nucleic acid can be introduced using the same or different systems. For example, a donor polynucleotide can be carried by a plasmid, while the Cas endonucleases can be carried by an AAV vector. Furthermore, the different vectors can be administered by the same or different routes (intramuscular injection, tail vein injection, other intravenous injection, intraperitoneal administration and/or intramuscular injection. The vectors can be delivered simultaneously or in any sequential order.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

In certain embodiments, the methods described herein involve introducing the composition described herein to hematopoietic stem/progenitor cells (HSPCs) or RBC precursors, thereby generating genetically modified HSPCs or RBC precursors. The genetically modified HSPCs and/or RBC precursors are given to a patient in a bone marrow transplant and the RBCs differentiate and mature in vivo. In some embodiments, the HSPCs are isolated from the peripheral blood following G-CSF-induced mobilization, and in others, the cells are isolated from human bone marrow or umbilical cord blood. In some aspects, the HSPCs are edited by treatment with a nuclease designed to knock out a specific gene or regulatory sequence. In other aspects, the HSPCs are modified with an engineered nuclease and a donor nucleic acid such that a wild type gene is inserted and expressed and/or an endogenous aberrant gene is corrected. In some embodiments, an engineered gene is inserted and expressed. In some embodiments, the modified HSPCs are administered to the patient following mild myeloablative pre-conditioning. In other aspects, the HSPCs are administered after full myeloablation such that following engraftment, a majority of the hematopoietic cells are derived from the newly engrafted modified HSC population.

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent components, methods, and means.

It is appreciated that the Summary and Abstract sections may set forth one or more, but not all exemplary embodiments of the present invention as contemplated by the inventor(s), and thus, are not intended to limit the present invention and the appended claims in any way.

### EXAMPLES

### Example 1

This example illustrates the generation of cell line models of SCD and thalassemia.

To generated a SCD cell line, the sickle mutation (Glu6Val, GAG to GTG) was introduced into the HBB gene in wild-type human umbilical cord blood-derived erythroid progenitor (HUDEP) cell line (HUDEP-2), which has the phenotype of adult erythroid cells with high level of β-globin expression and suppressed γ-globin expression. SpCas9 (Alt-R® S.p. HiFi Cas9 Nuclease V3, IDT), gRNA (SCD-gRNA (guide sequence see SEQ ID NO: 2)) and single-strand DNA (ssDNA) donor (SCD-ssDNA (SEQ ID NO: 58)) with homology arms flanking the gene were delivered by electroporation (Neon Transfection System) into the wild-type HUDEP-2 cells.

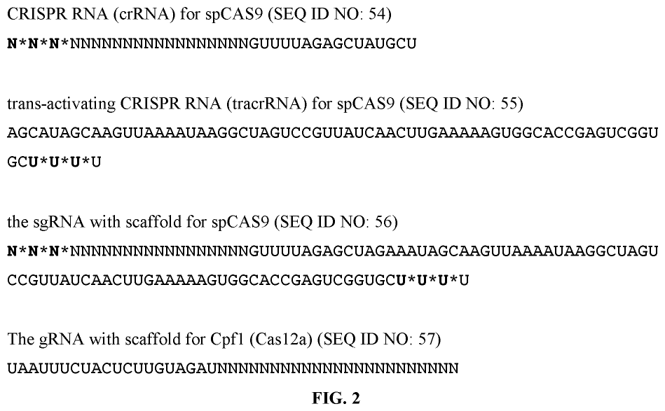

The scaffold of the sgRNA designed for Alt-R® S.p. HiFi Cas9 system is shown in FIG. 2 (SEQ ID NO:56). Nucleotides shown in bold are bases with 2′-O-Methylation modification, and the asterisks indicate phosphorothioate linkages. Chemical modifications on Alt-R CRISPR-Cas9 sgRNAs increase their stability, potency, and resistance against nuclease activity.

Five days after electroporation, single cells were sorted into 96-well plates by a BD FACS Melody™ cell sorter and cultured for 15 days. The genotype of each cell clone was determined by next generation of sequencing (NGS) (MiniSeq, Illumina). An HUDEP-2 clone homozygous for the sickle mutation was designated as the SCD cell line.

The β°/β° thalassemia cell line with 4 base-deletion in HBB exon 2 was generated to simulate frame-shift mutations in HBB exon 2. SpCas9 (Alt-R® S.p. HiFi Cas9 Nuclease V3, IDT) and gRNA β°-gRNA (guide sequence see SEQ ID NO: 60, which targets SEQ ID NO: 59) were delivered into wild-type HUDEP-2 cells by electroporation (Neon Transfection System). Five days after electroporation, single cells were sorted into 96-well plates by a BD FACS Melody™ cell sorter and cultured for 15 days. The genotype of each cell clone was determined by next generation sequencing (NGS) (Miniseq, Illumina); the clone homozygous for the—TTTG deletion was designated as the β°/β° cell line; lack of β-globin expression was confirmed by with intracellular staining and western blot for β-globin.

### Example 2

This example illustrates the design and test of gRNAs targeting the HBB gene for treating SCD and thalassemia.

To find a guide RNA that can efficiently target the HBB locus using SpCas9, the inventors designed a series of sgRNAs in the Table 1 below.

TABLE 1
gRNA sequences for targeting the HBB locus using SpCas9.

| | SEQ ID | | SEQ ID |
|---|---|---|---|
| Target sequence for HBB | NO: | Guide sequence | NO: |
| GTAACGGCAGACTTCTCCTCAGG | 1 | GUAACGGCAGACUUCUCCUC | 2 |
| GTAACGGCAGACTTCTCCTCAGG | 1 | UAACGGCAGACUUCUCCUC | 3 |
| GTAACGGCAGACTTCTCCTCAGG | 1 | AACGGCAGACUUCUCCUC | 4 |
| GTAACGGCAGACTTCTCCTCAGG | 1 | ACGGCAGACUUCUCCUC | 5 |
| GTAACGGCAGACTTCTCCTCAGG | 1 | CGGCAGACUUCUCCUC | 6 |
| GTAACGGCAGACTTCTCCTCAGG | 1 | GGCAGACUUCUCCUC | 7 |
| GTAACGGCAGACTTCTCCACAGG | 8 | GUAACGGCAGACUUCUCCAC | 9 |
| GTAACGGCAGACTTCTCCACAGG | 8 | UAACGGCAGACUUCUCCAC | 10 |
| GTAACGGCAGACTTCTCCACAGG | 8 | AACGGCAGACUUCUCCAC | 11 |
| GTAACGGCAGACTTCTCCACAGG | 8 | ACGGCAGACUUCUCCAC | 12 |
| GTAACGGCAGACTTCTCCACAGG | 8 | CGGCAGACUUCUCCAC | 13 |
| GTAACGGCAGACTTCTCCACAGG | 8 | GGCAGACUUCUCCAC | 14 |
| CACAGGAGTCAGATGCACCATGG | 15 | CACAGGAGUCAGAUGCACCA | 16 |
| CACAGGAGTCAGATGCACCATGG | 15 | ACAGGAGUCAGAUGCACCA | 17 |
| CACAGGAGTCAGATGCACCATGG | 15 | CAGGAGUCAGAUGCACCA | 18 |
| CACAGGAGTCAGATGCACCATGG | 15 | AGGAGUCAGAUGCACCA | 19 |
| CACAGGAGTCAGATGCACCATGG | 15 | GGAGUCAGAUGCACCA | 20 |
| CACAGGAGTCAGATGCACCATGG | 15 | GAGUCAGAUGCACCA | 21 |
| GCAACCTCAAACAGACACCATGG | 22 | GCAACCUCAAACAGACACCA | 23 |
| GCAACCTCAAACAGACACCATGG | 22 | CAACCUCAAACAGACACCA | 24 |
| GCAACCTCAAACAGACACCATGG | 22 | AACCUCAAACAGACACCA | 25 |
| GCAACCTCAAACAGACACCATGG | 22 | ACCUCAAACAGACACCA | 26 |
| GCAACCTCAAACAGACACCATGG | 22 | CCUCAAACAGACACCA | 27 |
| GCAACCTCAAACAGACACCATGG | 22 | CUCAAACAGACACCA | 28 |

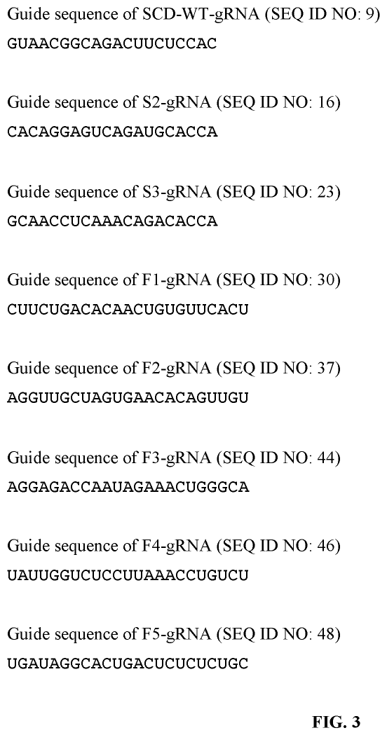

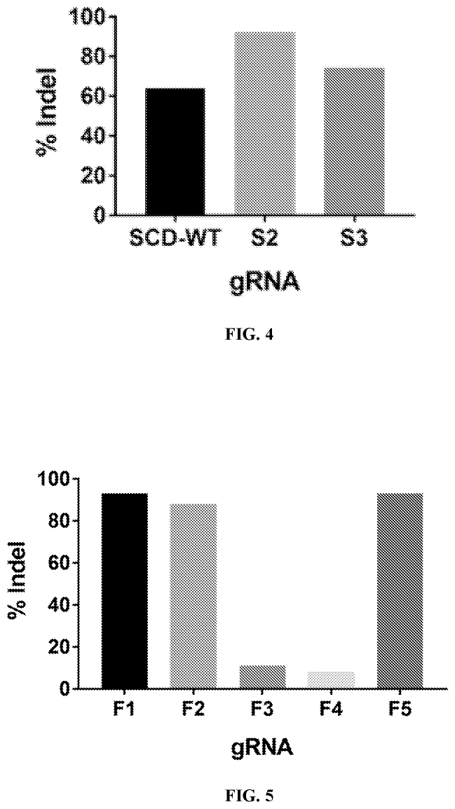

A set of SpCas9 gRNAs (SCD-WT-gRNA (guide sequence see SEQ ID NO: 9), S2-gRNA (guide sequence see SEQ ID NO: 16 and S3-gRNA (guide sequence see SEQ ID NO: 23), FIG. 3) targeting SCD HBB exon 1 were tested in the SCD cell line. SpCas9 (Alt-R® S.p. HiFi Cas9 Nuclease V3, IDT) and gRNA were delivered by electroporation (Neon Transfection System). Five days post electroporation, NGS analysis showed efficient cutting (>60% indel) by all three gRNAs (FIG. 4), suggesting they could be used to modify and repair the HBB gene.

The inventors also designed a series of gRNAs for targeting the HBB locus using Cpf1 (Cas12a), these gRNAs are listed in the Table 2 below.

TABLE 2
gRNA sequences for targeting the HBB locus using Cpf1.

| | SEQ | | SEQ |
|---|---|---|---|
| Target sequence for HBB | NO: | Guide sequence | NO: |
| TTTGCTTCTGACACAACTGTGTTCACT | 29 | CUUCUGACACAACUGUGUUCACU | 30 |
| TTTGCTTCTGACACAACTGTGTTCACT | 29 | CUUCUGACACAACUGUGUUCAC | 31 |
| TTTGCTTCTGACACAACTGTGTTCACT | 29 | CUUCUGACACAACUGUGUUCA | 32 |
| TTTGCTTCTGACACAACTGTGTTCACT | 29 | CUUCUGACACAACUGUGUUC | 33 |
| TTTGCTTCTGACACAACTGTGTTCACT | 29 | CUUCUGACACAACUGUGUU | 34 |
| TTTGCTTCTGACACAACTGTGTTCACT | 29 | CUUCUGACACAACUGUGU | 35 |
| TTTGAGGTTGCTAGTGAACACAGTTGT | 36 | AGGUUGCUAGUGAACACAGUUGU | 37 |
| TTTGAGGTTGCTAGTGAACACAGTTGT | 36 | AGGUUGCUAGUGAACACAGUUG | 38 |
| TTTGAGGTTGCTAGTGAACACAGTTGT | 36 | AGGUUGCUAGUGAACACAGUU | 39 |
| TTTGAGGTTGCTAGTGAACACAGTTGT | 36 | AGGUUGCUAGUGAACACAGU | 40 |
| TTTGAGGTTGCTAGTGAACACAGTTGT | 36 | AGGUUGCUAGUGAACACAG | 41 |
| TTTGAGGTTGCTAGTGAACACAGTTGT | 36 | AGGUUGCUAGUGAACACA | 42 |
| TTTAAGGAGACCAATAGAAACTGGGCA | 43 | AGGAGACCAAUAGAAACUGGGCA | 44 |
| TTTCTATTGGTCTCCTTAAACCTGTCT | 45 | UAUUGGUCUCCUUAAACCUGUCU | 46 |
| TTTCTGATAGGCACTGACTCTCTCTGC | 47 | UGAUAGGCACUGACUCUCUCUGC | 48 |
| TTTCTGATAGGCACTGACTCTCTCTGC | 47 | UGAUAGGCACUGACUCUCUCUG | 49 |
| TTTCTGATAGGCACTGACTCTCTCTGC | 47 | UGAUAGGCACUGACUCUCUCU | 50 |
| TTTCTGATAGGCACTGACTCTCTCTGC | 47 | UGAUAGGCACUGACUCUCUC | 51 |
| TTTCTGATAGGCACTGACTCTCTCTGC | 47 | UGAUAGGCACUGACUCUCU | 52 |
| TTTCTGATAGGCACTGACTCTCTCTGC | 47 | UGAUAGGCACUGACUCUC | 53 |

A set of cpf1-gRNAs targeting HBB exon 1 (F1 (guide sequence see SEQ ID NO: 30) and F2 (guide sequence see SEQ ID NO: 37)) and HBB intron 1 (F3 (guide sequence see SEQ ID NO: 44), F4 (guide sequence see SEQ ID NO: 46) and F5 (guide sequence see SEQ ID NO: 48) were tested in the SCD cell line. gRNAs with scaffold for Cpf1 (see SEQ ID NO: 57) and cpf1 (Alt-R® A.s. Cas12a (Cpf1) Ultra, IDT) were delivered into the SCD cell line by electroporation (Neon Transfection System). Five days post electroporation, NGS analysis showed efficient cutting by F1, F2 and F5 (FIG. 5), suggesting these gRNAs may be used to modify and repair the HBB gene.

### Example 3

This example illustrates the HDR-based repair of the HBB gene using optimized guide RNA.

Using gRNAs that target exon 1, if a corrective DNA donor with homology arms is provided, it is feasible to invoke the homology-directed repair (HDR) pathway to correct the sickle mutation. For HBB alleles that contain loss-of-function mutations (e.g. those cause beta-thalassemia) located downstream of the exon 1 gRNA cleavage sites, it is feasible to use those gRNAs and HDR to integrate a normal copy of the HBB gene to restore expression of normal b-globin.

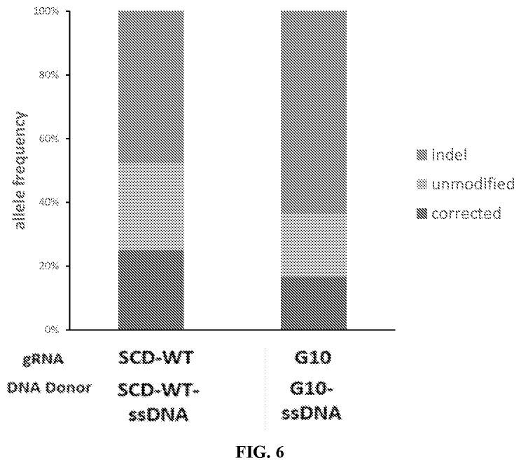

To repair the sickle mutation, gRNAs that cleave close to the mutation (SCD-WT-gRNA (guide sequence see SEQ ID NO: 9) or G10-gRNA (guide sequence see SEQ ID NO: 62, which targets SEQ ID NO:61) (Dever, D. et al. Nature (2016) 539, 384-389) CRISPR/Cas9 β-globin gene targeting in human haematopoietic stem cells. Nature 539, 384-389 (2016).)) were co-delivered, by electroporation (Neon Transfection System), into the SCD cell line with SpCas9 (Alt-R® S.p. HiFi Cas9 Nuclease V3, IDT) and their respective ssDNA donors (SCD-WT-ssDNA (SEQ ID NO: 63) or G10-ssDNA (SEQ ID NO: 64)) that contain homology arms flanking the site of cleavage. The efficiency of sickle mutation correction was analyzed by NGS at 5 days post electroporation (FIG. 6). Correction of the sickle mutation was observed in 23% and 14% of the alleles in SCD-WT-gRNA and G10 gRNA-treated cells, respectively.

### Example 4

This example illustrates the non-homology-based repair of the HBB gene using optimized guide RNA.

Using gRNAs that target intron 1 of HBB, it is possible to use non-homology-based repair to integrate a normal copy of the HBB gene (without homology arms, but with a splicing acceptor site to ensure correct splicing with the native HBB exon 1) into intron 1, which restores expression of normal b-globin from the disease allele. The non-homology-based repair may occur at a higher frequency than HDR.

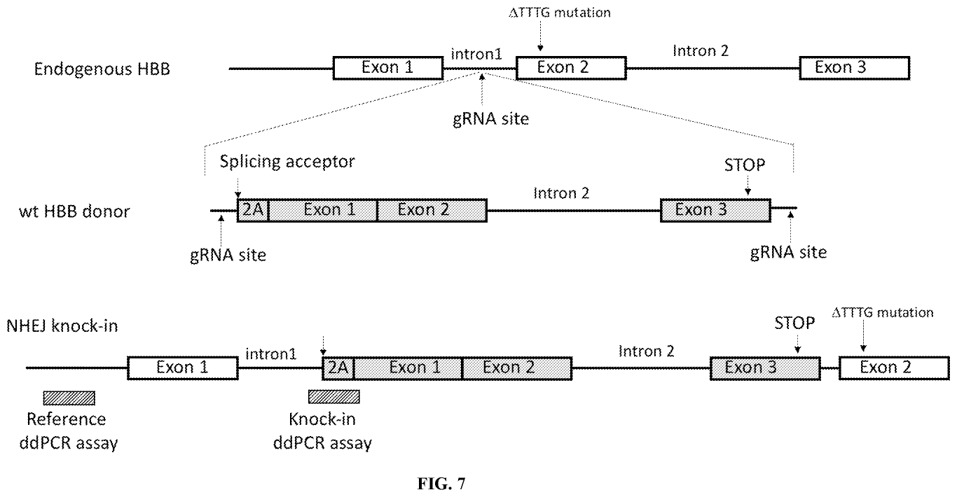

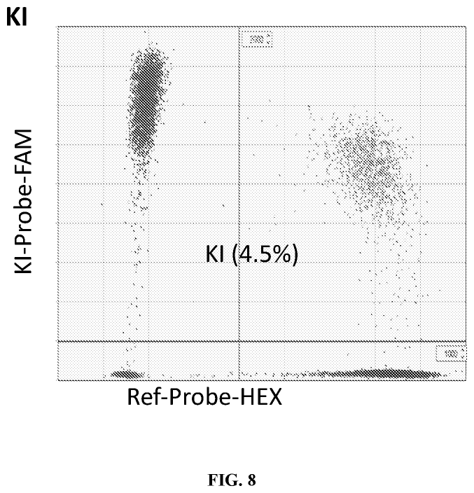

Cpf1, gRNA HBBcpf1-5 (F5) (cpf1/F5 RNP) and a linear dsDNA donor (consists of splicing acceptor site, HBB exon 1, exon 2, intron 2 and exon 3) (SEQ ID NO: 65) were delivered into the β°/β° cell line by electroporation (Neon Transfection System). Five days post electroporation, knock-in efficiency was determined by a digital droplet PCR (ddPCR) assay (ddPCR™ NHEJ Genome Edit Detection Assays, Bio-Rad), which specifically amplifies the region that spans the 5′ junction of the donor insertion (FIG. 7). ddPCR analysis showed knock-in efficiency of 4.5% (FIG. 8).

1. A guide RNA or a nucleic acid encoding the same, which targets a sequence as shown in any of SEQ ID NOs: 29, 36 and 47 in a beta-globin gene.

2. The guide RNA or nucleic acid of claim 1, wherein the guide RNA comprises a polynucleotide sequence having at least 95% identity to any of SEQ ID NOS: 30-35, 37-42 and 48-53.

3. The guide RNA or nucleic acid of claim 1, wherein the guide RNA comprises a polynucleotide sequence of any of SEQ ID NOS: 30-35, 37-42 and 48-53.

4-7. (canceled)

8. The guide RNA or nucleic acid of claim 1, wherein the guide RNA is for Cpf1.

9. A composition comprising
a CRISPR/Cas nuclease or a nucleic acid encoding the same; and
the guide RNA or a nucleic acid encoding the same according to claim 1,
wherein the CRISPR/Cas nuclease is associated with the guide RNA and is capable of cleaving the beta-globin gene.

10. The composition of claim 9, wherein the CRISPR/Cas nuclease is a Cpf1 nuclease.

11. The composition of claim 9, further comprising a donor nucleic acid, wherein the donor nucleic acid comprises a transgene encoding a wildtype beta-globin polypeptide.

12. (canceled)

13. The composition of claim 11, wherein the donor nucleic acid is a single-strand DNA or double-strand DNA.

14. An isolated mammalian cell comprising the composition of claim 9.

15. The isolated mammalian cell of claim 14, which is a stem cell, wherein the stem cell is a hematopoietic stem/progenitor cell (HSPC).

16. (canceled)

17. The isolated mammalian cell of claim 14, which is obtained from a subject having beta-thalassemia or sickle cell disease.

18. The isolated mammalian cell of claim 14, further comprising a transgene encoding a wildtype beta-globin polypeptide.

19. A method of modifying an isolated mammalian cell, the method comprising introducing to the mammalian cell the composition of claim 9, wherein the CRISPR/Cas nuclease cleaves the beta-globin gene in the mammalian cell.

20. The method of claim 19, wherein the mammalian cell is a stem cell, and the stem cell is a HSPC.

21. (canceled)

22. The method of claim 19, wherein the mammalian cell is obtained from a subject having beta-thalassemia or sickle cell disease, the method further comprising introducing to the mammalian cell a nucleic acid comprising a transgene encoding a wildtype beta-globin polypeptide such that the transgene is inserted to the target site.

23. The method of claim 19, wherein the nucleic acid is a single-strand DNA or double-strand DNA.

24. The method of claim 19, wherein the nucleic acid is contained in a virus vector, and the virus is adeno-associated virus (AAV).

25-28. (canceled)

29. A method of treating beta-thalassemia or SCD in a subject, the method comprising administering to the subject the mammalian cell of claim 14.

30. The method of claim 29, wherein the mammalian cell is a stem cell obtained from the subject, and the stem cell is a HSPC.

31. (canceled)

32. The method of claim 30, wherein a transgene encoding a wildtype beta-globin is inserted to the target site in the HSPC, thereby generating a modified HSPC, the method further comprising administering the modified HSPC to the subject.

33. (canceled)

34. A method of treating beta-thalassemia or SCD comprising in a subject, the method comprising administering to the subject the composition of claim 9, wherein the CRISPR/Cas nuclease cleaves the beta-globin gene in a cell of the subject.


SEQUENCE LISTING (65 sequences)

SEQ ID NO: 1 - DNA, 23 bp, Homo sapiens
gtaacggcagacttctcctcagg

SEQ ID NO: 2 - RNA, 20 nt, Artificial Sequence
  Synthetic
guaacggcagacuucuccuc

SEQ ID NO: 3 - RNA, 19 nt, Artificial Sequence
  Synthetic
uaacggcagacuucuccuc

SEQ ID NO: 4 - RNA, 18 nt, Artificial Sequence
  Synthetic
aacggcagacuucuccuc

SEQ ID NO: 5 - RNA, 17 nt, Artificial Sequence
  Synthetic
acggcagacuucuccuc

SEQ ID NO: 6 - RNA, 16 nt, Artificial Sequence
  Synthetic
cggcagacuucuccuc

SEQ ID NO: 7 - RNA, 15 nt, Artificial Sequence
  Synthetic
ggcagacuucuccuc

SEQ ID NO: 8 - DNA, 23 bp, Homo sapiens
gtaacggcagacttctccacagg

SEQ ID NO: 9 - RNA, 20 nt, Artificial Sequence
  Synthetic
guaacggcagacuucuccac

SEQ ID NO: 10 - RNA, 19 nt, Artificial Sequence
  Synthetic
uaacggcagacuucuccac

SEQ ID NO: 11 - RNA, 18 nt, Artificial Sequence
  Synthetic
aacggcagacuucuccac

SEQ ID NO: 12 - RNA, 17 nt, Artificial Sequence
  Synthetic
acggcagacuucuccac

SEQ ID NO: 13 - RNA, 16 nt, Artificial Sequence
  Synthetic
cggcagacuucuccac

SEQ ID NO: 14 - RNA, 15 nt, Artificial Sequence
  Synthetic
ggcagacuucuccac

SEQ ID NO: 15 - DNA, 23 bp, Homo sapiens
cacaggagtcagatgcaccatgg

SEQ ID NO: 16 - RNA, 20 nt, Artificial Sequence
  Synthetic
cacaggagucagaugcacca

SEQ ID NO: 17 - RNA, 19 nt, Artificial Sequence
  Synthetic
acaggagucagaugcacca

SEQ ID NO: 18 - RNA, 18 nt, Artificial Sequence
  Synthetic
caggagucagaugcacca

SEQ ID NO: 19 - RNA, 17 nt, Artificial Sequence
  Synthetic
aggagucagaugcacca

SEQ ID NO: 20 - RNA, 16 nt, Artificial Sequence
  Synthetic
ggagucagaugcacca

SEQ ID NO: 21 - RNA, 15 nt, Artificial Sequence
  Synthetic
gagucagaugcacca

SEQ ID NO: 22 - DNA, 23 bp, Homo sapiens
gcaacctcaaacagacaccatgg

SEQ ID NO: 23 - RNA, 20 nt, Artificial Sequence
  Synthetic
gcaaccucaaacagacacca

SEQ ID NO: 24 - RNA, 19 nt, Artificial Sequence
  Synthetic
caaccucaaacagacacca

SEQ ID NO: 25 - RNA, 18 nt, Artificial Sequence
  Synthetic
aaccucaaacagacacca

SEQ ID NO: 26 - RNA, 17 nt, Artificial Sequence
  Synthetic
accucaaacagacacca

SEQ ID NO: 27 - RNA, 16 nt, Artificial Sequence
  Synthetic
ccucaaacagacacca

SEQ ID NO: 28 - RNA, 15 nt, Artificial Sequence
  Synthetic
cucaaacagacacca

SEQ ID NO: 29 - DNA, 27 bp, Homo sapiens
tttgcttctgacacaactgtgttcact

SEQ ID NO: 30 - RNA, 23 nt, Artificial Sequence
  Synthetic
cuucugacacaacuguguucacu

SEQ ID NO: 31 - RNA, 22 nt, Artificial Sequence
  Synthetic
cuucugacacaacuguguucac

SEQ ID NO: 32 - RNA, 21 nt, Artificial Sequence
  Synthetic
cuucugacacaacuguguuca

SEQ ID NO: 33 - RNA, 20 nt, Artificial Sequence
  Synthetic
cuucugacacaacuguguuc

SEQ ID NO: 34 - RNA, 19 nt, Artificial Sequence
  Synthetic
cuucugacacaacuguguu

SEQ ID NO: 35 - RNA, 18 nt, Artificial Sequence
  Synthetic
cuucugacacaacugugu

SEQ ID NO: 36 - DNA, 27 bp, Homo sapiens
tttgaggttgctagtgaacacagttgt

SEQ ID NO: 37 - RNA, 23 nt, Artificial Sequence
  Synthetic
agguugcuagugaacacaguugu

SEQ ID NO: 38 - RNA, 22 nt, Artificial Sequence
  Synthetic
agguugcuagugaacacaguug

SEQ ID NO: 39 - RNA, 21 nt, Artificial Sequence
  Synthetic
agguugcuagugaacacaguu

SEQ ID NO: 40 - RNA, 20 nt, Artificial Sequence
  Synthetic
agguugcuagugaacacagu

SEQ ID NO: 41 - RNA, 19 nt, Artificial Sequence
  Synthetic
agguugcuagugaacacag

SEQ ID NO: 42 - RNA, 18 nt, Artificial Sequence
  Synthetic
agguugcuagugaacaca

SEQ ID NO: 43 - DNA, 27 bp, Homo sapiens
tttaaggagaccaatagaaactgggca

SEQ ID NO: 44 - RNA, 23 nt, Artificial Sequence
  Synthetic
aggagaccaauagaaacugggca

SEQ ID NO: 45 - DNA, 27 bp, Homo sapiens
tttctattggtctccttaaacctgtct

SEQ ID NO: 46 - RNA, 23 nt, Artificial Sequence
  Synthetic
uauuggucuccuuaaaccugucu

SEQ ID NO: 47 - DNA, 27 bp, Homo sapiens
tttctgataggcactgactctctctgc

SEQ ID NO: 48 - RNA, 23 nt, Artificial Sequence
  Synthetic
ugauaggcacugacucucucugc

SEQ ID NO: 49 - RNA, 22 nt, Artificial Sequence
  Synthetic
ugauaggcacugacucucucug

SEQ ID NO: 50 - RNA, 21 nt, Artificial Sequence
  Synthetic
ugauaggcacugacucucucu

SEQ ID NO: 51 - RNA, 20 nt, Artificial Sequence
  Synthetic
ugauaggcacugacucucuc

SEQ ID NO: 52 - RNA, 19 nt, Artificial Sequence
  Synthetic
ugauaggcacugacucucu

SEQ ID NO: 53 - RNA, 18 nt, Artificial Sequence
  Synthetic
ugauaggcacugacucuc

SEQ ID NO: 54 - RNA, 36 nt, Artificial Sequence
  Synthetic
  misc_feature (1)..(20): n can be a, u, g or c.
nnnnnnnnnnnnnnnnnnnnguuuuagagcuaugcu

SEQ ID NO: 55 - RNA, 67 nt, Artificial Sequence
  Synthetic
agcauagcaaguuaaaauaaggcuaguccguuaucaacuugaaaaaguggcaccgagucggugcuuu

SEQ ID NO: 56 - RNA, 99 nt, Artificial Sequence
  Synthetic
  misc_feature (1)..(20): n can be a, u, g or c.
nnnnnnnnnnnnnnnnnnnnguuuuagagcuagaaauagcaaguuaaaauaaggcuaguccguuaucaacuugaaaaaguggcaccgagucggugcuuu

SEQ ID NO: 57 - RNA, 43 nt, Artificial Sequence
  Synthetic
  misc_feature (21)..(43): n can be a, u, g or c.
uaauuucuacucuuguagaunnnnnnnnnnnnnnnnnnnnnnn

SEQ ID NO: 58 - DNA, 185 bp, Artificial Sequence
  Synthetic
cagggcagagccatctattgcttacatttgcttctgacacaactgtgttcactagcaacctcaaacagacaccatggtgcatctgactcctgtggaaaagtctgccgttactgccctgtggggcaaggtgaacgtggatgaagttggtggtgaggccctgggcaggttggtatcaaggttacaag

SEQ ID NO: 59 - RNA, 23 nt, Homo sapiens
aggaguggacagauccccaaagg

SEQ ID NO: 60 - RNA, 21 nt, Artificial Sequence
  Synthetic
aggaguggacagauccccaaa

SEQ ID NO: 61 - DNA, 23 bp, Homo sapiens
cttgccccacagggcagtaacgg

SEQ ID NO: 62 - DNA, 20 bp, Artificial Sequence
  Synthetic
cuugccccacagggcaguaa

SEQ ID NO: 63 - DNA, 185 bp, Artificial Sequence
  Synthetic
cagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaag

SEQ ID NO: 64 - DNA, 185 bp, Artificial Sequence
  Synthetic
attgcttacatttgcttctgacacaactgtgttcactagcaacctcaaacagacaccatggtgcatctgactcctgaggagaagtctgccgtcactgccctgtggggcaaggtgaacgtggatgaagttggtggtgaggccctgggcaggttggtatcaaggttacaagacaggtttaaggagac

SEQ ID NO: 65 - DNA, 1581 bp, Artificial Sequence
  Synthetic
gcagagagagtcagtgcctatcagaaatctctctgcctattggtctattttcccacccttagctggaagcggagctactaacttcagcctgctgaagcaggctggagacgtggaggagaaccctggagtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatggcagagagagtcagtgcctatcagaaa